(19) United States
(12) Patent Application Publication
(10) Pub. No.: US 2011/0230946 A1
(43) Pub. Date: Sep. 22, 2011

(54) EASY MARKER PLACEMENT BALLOON MOLD

(75) Inventors: DARA J. BUTCHER, Temecula, CA (US); CARLOS A. ARIPEZ, Murrieta, CA (US)

(73) Assignee: ABBOTT LABORATORIES, Santa Clara, CA (US)

(21) Appl. No.: 12/725,063

(22) Filed: Mar. 16, 2010

(51) Int. Cl. A61F 2/84; A61M 25/10; B29C 49/78; B28B 11/08

(52) U.S. Cl. 623/1.11; 604/103.08; 425/173; 264/293

(57) ABSTRACT

A catheter balloon is formed from a mold process in which raised dimples or shallow divots are formed on the outer surface using indentations or raised beads on the mold surface. The dimples or divots are located at the transition between the body portion of the catheter balloon and the neck or taper section. The presence of the dimples or divots serves the dual purpose of identifying the transition between the two regions for placing a visual marker on the balloon to be used in positioning the balloon, and also to assist in the retention of a vascular stent on the balloon.

### BACKGROUND

This invention generally relates to intravascular balloon catheters such as those used in percutaneous transluminal coronary angioplasty (PTCA) and stent delivery, and more particularly to a catheter balloon and mold for creating a balloon that permits reliable securement of positioning markers and stents.

PTCA is a widely used procedure for the treatment of coronary heart disease. In this procedure, a balloon dilatation catheter is advanced into the patient's coronary artery and the balloon on the catheter is inflated within the stenotic region of the patient's artery to open up the arterial passageway and thereby increase the blood flow there through. To facilitate the advancement of the dilatation catheter into the patient's coronary artery, a guiding catheter having a preshaped distal tip is first percutaneously introduced into the cardiovascular system of a patient by the Seldinger technique or other method through the brachial or femoral arteries.

The catheter is advanced until the preshaped distal tip of the guiding catheter is disposed within the aorta adjacent the ostium of the desired coronary artery, and the distal tip of the guiding catheter is then maneuvered into the ostium. A balloon dilatation catheter may then be advanced through the guiding catheter into the patient's coronary artery over a guidewire until the balloon on the catheter is disposed within the stenotic region of the patient's artery. The balloon is inflated to open up the arterial passageway and increase the blood flow through the artery. Generally, the inflated diameter of the balloon is approximately the same diameter as the native diameter of the body lumen being dilated so as to complete the dilatation but not over expand the artery wall. After the balloon is finally deflated, blood flow resumes through the dilated artery and the dilatation catheter can be removed.

In a large number of angioplasty procedures, there may be a restenosis, i.e. reformation of the arterial plaque. To reduce the restenosis rate and to strengthen the dilated area, physicians may implant an intravascular prosthesis or “stent” inside the artery at the site of the lesion. Stents may also be used to repair vessels having an intimal flap or dissection or to generally strengthen a weakened section of a vessel. Stents are usually delivered to a desired location within a coronary artery in a contracted condition on a balloon of a catheter which is similar in many respects to a balloon angioplasty catheter, and expanded to a larger diameter by expansion of the balloon. The balloon is then deflated to remove the catheter and the stent is left in place within the artery at the site of the dilated lesion.

To accurately place the balloon at the desired location, visual markers on the balloon are utilized that are read by machines outside the body. For example, in the case where a balloon catheter is used with an fluoroscope, the radiopaque marker may be observed visually on a screen while the procedure is taking place. In many cases, the markers must be precisely located to ensure accurate placement of the balloon in the affected area. When stents are being deployed the location of the beginning and ending point of the stent can be crucial to the success of the procedure. In such cases, it is preferred that the markers be located very specifically at the junction of the body portion of the balloon with the neck portion. However, it is also important that the marker not be located on the neck portion of the balloon. Unfortunately, the manufacturing process does not readily lend itself to a precise determination as to where to apply the marker such that it is at the extreme end of the working portion of the balloon but does not extend to the neck portion.

### SUMMARY OF THE INVENTION

The present invention addresses the problem above by using a modified mold to create visual locators on the balloon that identify the proper position of the marker. A ring of visual locators on the balloon can be created by putting indentions or beads on the mold used to form the balloon, causing the balloon to have raised dimples in the case of indentations or shallow cavities in the case of beads on the working portion of the balloon right before the taper or neck portion. Henceforth the application shall refer to the visual markers as dimples but it is to be understood that cavities or divots would serve the same purpose and are considered part of the invention. The raised dimples enable the manufacturing operators who are tasked with placing the visual markers on the balloon to quickly and easily locate the visual markers precisely before the neck region but in the working area of the balloon every time. This also aids in the placement of stents that are mounted on the balloon in procedures that use this feature. The raised dimples also can assist in the retention of the stent on the balloon as it passes through the patient's vascular, where the raised dimples provide a resistance against slippage of the stent off of the balloon.

### BRIEF DESCRIPTION OF THE DRAWINGS

FIG. 1 is an elevated view partially in section of a balloon catheter of the present invention;

FIG. 2 is a transverse cross sectional view of the balloon catheter of FIG. 1 taken along lines 2-2;

FIG. 3 is a transverse cross sectional view of the balloon catheter of FIG. 1 taken along lines 3-3;

FIG. 4 is an enlarged view of the balloon catheter of FIG. 1 with a vascular stent mounted thereon;

FIG. 5 is an enlarged view of the stent of FIG. 4 disposed in a patient's vascular after removal of the balloon;

FIG. 6. is an even more enlarged view of the distal end of the balloon and stent of FIG. 4 showing the raised dimples;

FIG. 7 is a cut-away view of a mold for forming the balloon of the present invention and a balloon tubing prior to forming; and

FIG. 8 is a cut-away view of the mold of FIG. 7 after pressurization and heating to form the balloon of the present invention.

### DETAILED DESCRIPTION OF THE PREFERRED EMBODIMENTS

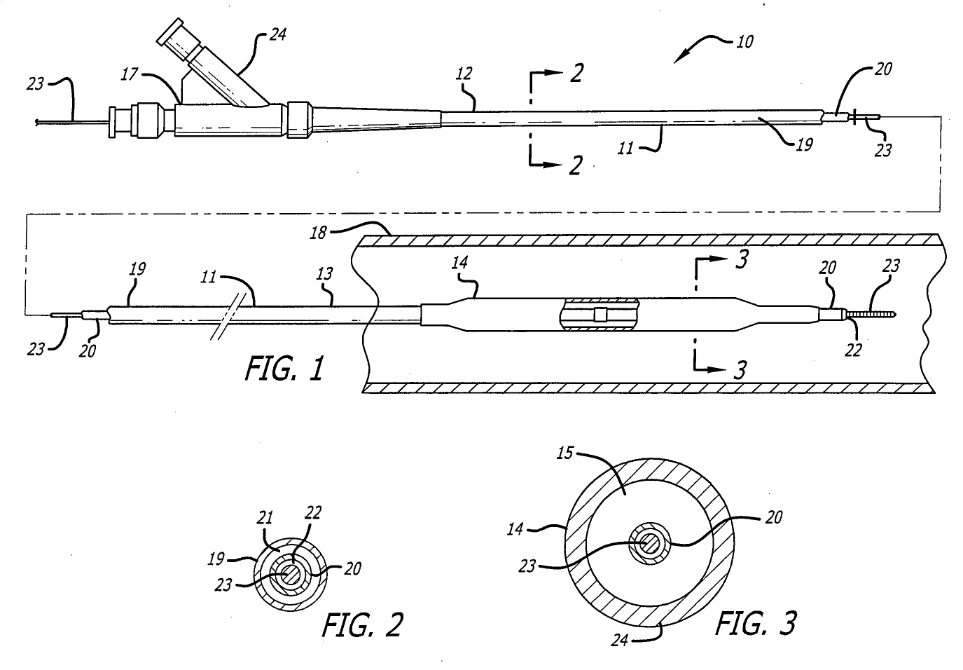

FIG. 1 shows a balloon catheter that can be used to illustrate the features of the invention. The catheter 10 of the invention generally comprises an elongated catheter shaft 11 having a proximal section 12, a distal section 13, an inflatable balloon 14 on the distal section 13 of the catheter shaft 11, and an adapter 17 mounted on the proximal section 12 of shaft 11. In FIG. 1, the catheter 10 is illustrated within a greatly enlarged view of a patient's body lumen 18, prior to expansion of the balloon 14, adjacent the tissue to be injected with therapeutic agents.

In the embodiment illustrated in FIG. 1, the catheter shaft 11 has an outer tubular member 19 and an inner tubular member 20 disposed within the outer tubular member and defining, with the outer tubular member, inflation lumen 21. Inflation lumen 21 is in fluid communication with the interior chamber 15 of the inflatable balloon 14. The inner tubular member 20 has an inner lumen 22 extending therein which is configured to slidably receive a guidewire 23 suitable for advancement through a patient's coronary arteries. The distal extremity of the inflatable balloon 14 is sealingly secured to the distal extremity of the inner tubular member 20 and the proximal extremity of the balloon is sealingly secured to the distal extremity of the outer tubular member 19.

FIGS. 2 and 3 show transverse cross sections of the catheter shaft 11 and balloon 14, respectively, illustrating the guidewire receiving lumen 22 of the guidewire's inner tubular member 20 and inflation lumen 21 leading to the balloon interior 15. The balloon 14 can be inflated by a fluid such as air, saline, or other fluid that is introduced at the port in the side arm 25 into inflation lumen 21 contained in the catheter shaft 11, or by other means, such as from a passageway formed between the outside of the catheter shaft 11 and the member forming the balloon 14, depending on the particular design of the catheter. The details and mechanics of the mode of inflating the balloon vary according to the specific design of the catheter, and are omitted from the present discussion.

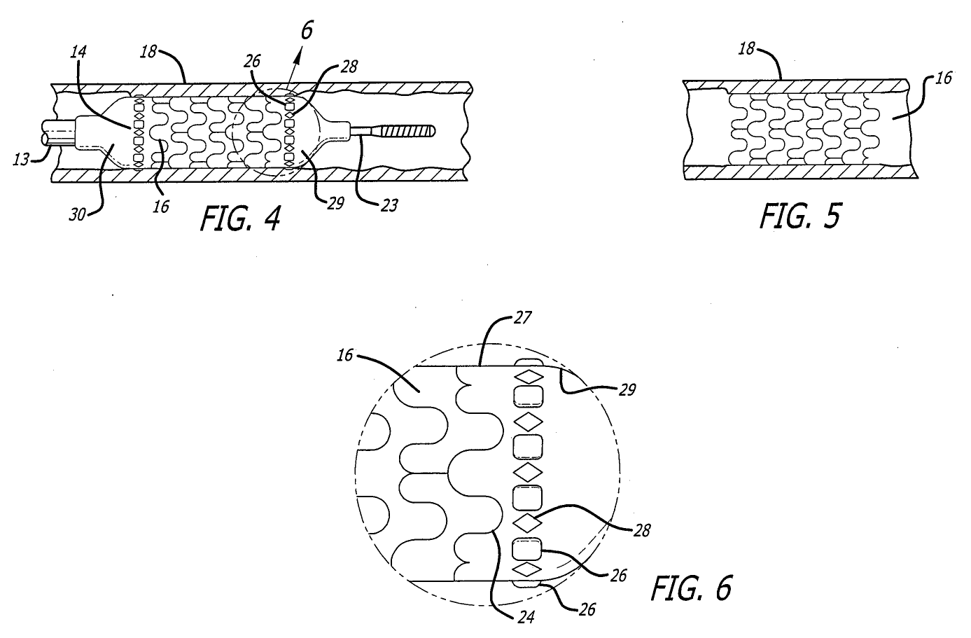

FIG. 4 illustrates an embodiment of the catheter of FIG. 1 with a vascular stent 16 mounted thereon. The stent 16 can be made in many ways. One method of making the stent is to cut a thin-walled tubular member, such as stainless steel tubing to remove portions of the tubing in the desired pattern for the stent, leaving relatively untouched the portions of the metallic tubing which are to form the stent 16. The stent 16 also can be made from other metal alloys such as tantalum, nickel-titanium, cobalt-chromium, titanium, shape memory and superelastic alloys, and the Nobel metals such as gold or platinum. It is preferred to cut the tubing in the desired pattern by means of a machine-controlled laser as is well known in the art. Stents function to hold open a segment of a blood vessel or other body lumen such as a renal or coronary artery. At present, there are numerous commercial stents being marketed throughout the world. While some of these stents are flexible and have the appropriate radial rigidity needed to hold open a vessel or artery, there typically is a tradeoff between flexibility and radial strength and the ability to tightly compress or crimp the stent onto a catheter so that it does not move relative to the catheter or dislodge prematurely prior to controlled implantation in a vessel. Currently, to secure a stent 16 on a balloon 14, after the stent is crimped onto the deflated balloon such that the balloon partially protrudes through the stent struts. During this process, the balloon and stent are placed in a heated mold and pressurized. The balloon protrusions then acts as holds to secure the stent in place.

In a typical procedure to implant stent 16, the guide wire 23 is advanced through the patient's vascular system by well known methods so that the distal end of the guide wire is advanced past the location for the placement of the stent in the body lumen 18. Prior to implanting the stent 16, the cardiologist may wish to perform an angioplasty procedure or other procedure (i.e., atherectomy) in order to open the vessel and remodel the diseased area. Thereafter, the stent delivery catheter assembly 10 is advanced over the guide wire 23 so that the stent 16 is positioned in the target area. The balloon 14 is inflated so that it expands radially outwardly and in turn expands the stent 16 radially outwardly until the stent 16 bears against the vessel wall of the body lumen 18. The balloon 14 is then deflated and the catheter withdrawn from the patient's vascular system, leaving the stent 16 in place to dilate the body lumen. The guide wire 23 typically is left in the lumen for post-dilatation procedures, if any, and subsequently is withdrawn from the patient's vascular system. As depicted in FIG. 4, the balloon 14 is fully inflated with the stent 16 expanded and pressed against the vessel wall, and in FIG. 5, the implanted stent 16 remains in the vessel after the balloon has been deflated and the catheter assembly and guide wire have been withdrawn from the patient.

FIG. 6 illustrates a close up section of the balloon 14 showing raised dimples 26 in a circumferential ring at the juncture of the body section 27 of the balloon 14 and the onset of the neck section 29. Again, the dimples can be replaced with shallow divots or cavities as long as the structural integrity of the balloon is not compromised. The dimples 26 are shown as rectangular in shape although other shapes are possible. The dimples 26 form a ring or perimeter around the balloon 14 defining the end of the balloon body or working portion 27 of the balloon, and consequently coinciding with the edge 24 of the stent 16. For example, in a first preferred embodiment the dimples 26 are located one millimeter from the beginning of the taper or neck section of the balloon. Between dimples 26 a radiopaque marker 28 is secured to the balloon that can be used to locate both the balloon 14 and the stent 16. It is to be understood that a similar ring of dimples 26 will ordinarily be formed at the proximal end of the working section 27 of the balloon 14 where it tapers into the proximal neck portion 30. The markers 28 (shown as diamond shaped but can be any shape or size) are observed under the fluoroscope or other means and can be used to precisely locate the catheter, the balloon 14, and the stent 16. The dimples 26 also ensure that the markers 28 are not placed by the distal seal operators at the taper sections of the balloons. The raised dimples 26 can also assist in stent retention, as the perimeter of raised dimples can assist the anchoring of the stent 16 as it is mounted on the balloon 14 and passed through the patient's vascular system. That is, the dimples 26 act as a retaining barrier that abuts the stent to keep it positioned properly on the balloon.

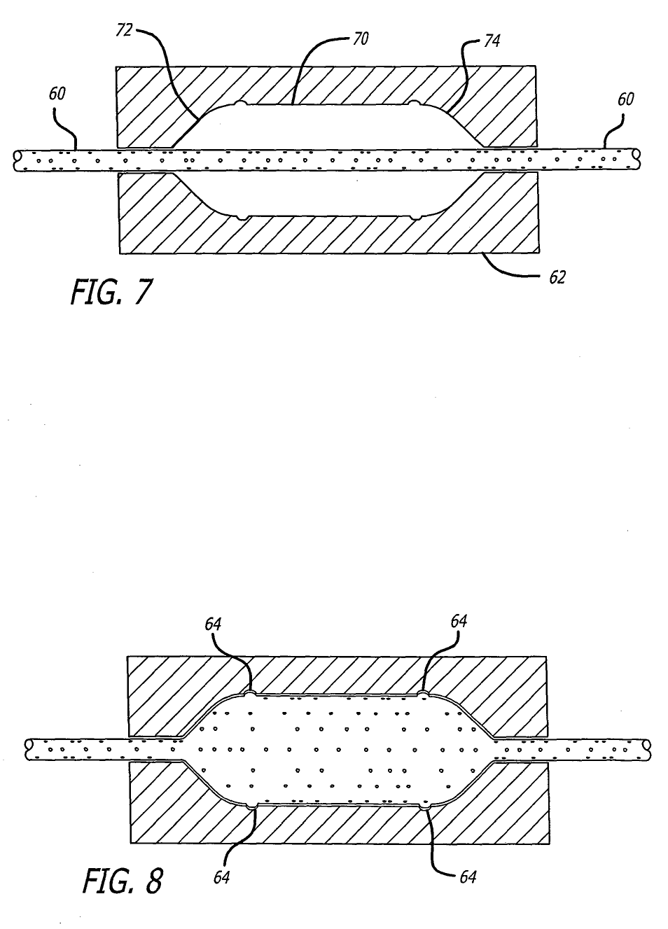

The balloon 14 is formed using conventional balloon technologies, such as blow molding as illustrated in FIGS. 7 and 8. A tube 60 of balloon material is inserted into a mold 62 having the desired balloon shape. The mold 62 has a constant radius wall 70 and an increasing radial section 72 at a first end and a decreasing radial section 74 at a second end, and further includes a series of cavities/indentations (or beads) 64 on the constant radius wall section at the transition to the radially increasing and decreasing portions. The indentations 64 fill with balloon material as the tube is expanded and heated to form a balloon with the desired raised dimples at the edges of the working section of the balloon 14. Alternatively, the beads form a slight cavity in the balloon producing a visual identifier of the edge of the working section. The balloon material is maintained in the heated and pressurized state until the balloon is formed to cause the tubing 60 to expand to the final shape within the mold 62, including the formation of the dimples or cavities 26 in the indentation. This will result in a balloon that includes the rings of dimples 26 shown in FIGS. 4 and 6.

While particular forms of the invention have been illustrated and described, it will be apparent to those skilled in the art that various modifications can be made without departing from the spirit and scope of the invention. Accordingly, it is not intended that the invention be limited except by the appended claims.

What is claimed:

1. A catheter balloon, comprising:
a working body section and a proximal and a distal taper section; and
a ring of dimples encircling the balloon and demarking the transition between the working body section and an adjacent taper section.

2. The catheter balloon of claim 1, further comprising a visual marker disposed at the ring of dimples where the visual marker can be viewed from outside a patient's body when the catheter balloon is located within the patient's body.

3. The catheter balloon of claim 1, further comprising a second ring of dimples encircling the balloon and demarking the transition between the working body and another adjacent taper section.

4. The catheter balloon of claim 1, further comprising a stent mounted on the balloon, wherein the ring of dimples cooperates to retain the stent on the balloon.

5. The catheter balloon of claim 1, wherein the balloon is formed by blow molding.

6. A mold for a catheter balloon, comprising:
a first inner wall defining a constant radius void for forming a working portion of the balloon, second and third inner walls defining first and second neck portions of the balloon, and a ring of indentions located at a transition between the first wall and one of the second and third walls to form dimples on an outer surface of the balloon on the working portion adjacent the one of the second and third walls.

7. The mold of claim 6, further comprising a second ring of indentations located at a transition between the first wall and another of the second and third walls to form dimples on the outer surface of the balloon on the working portion adjacent the another one of the second and third walls.

8. A method for locating a positioning marker on a catheter balloon, comprising:
providing a mold for a balloon having an inner wall defining a shape of a balloon;
positioning indentions on the mold to create dimples on an outer surface of the balloon where the location of the dimples coincides with a desired location of the positioning marker; and
incorporating a positioning marker in the balloon using the dimples on the outer surface to place the positioning marker at the desired location.

9. A catheter balloon, comprising:
a working body section and a proximal and a distal taper section; and
a ring of divots encircling the balloon and demarking the transition between the working body section and an adjacent taper section.

10. The catheter balloon of claim 9, further comprising a visual marker disposed at the ring of divots where the visual marker can be viewed from outside a patient's body when the catheter balloon is located within the patient's body.

11. The catheter balloon of claim 9, further comprising a second ring of divots encircling the balloon and demarking the transition between the working body and another adjacent taper section.

12. The catheter balloon of claim 9, wherein the balloon is formed by blow molding.
